(19) United States
(12) Patent Application Publication
(10) Pub. No.: US 2019/0117093 A1
(43) Pub. Date: Apr. 25, 2019

(54) METHOD AND DEVICE FOR MEASURING BLOOD PRESSURE

(71) Applicant: BOE TECHNOLOGY GROUP CO., LTD., Beijing (CN)

(72) Inventors: Hui DU, Beijing (CN); Yang HAN, Beijing (CL); Hailan JIN, Beijing (CN); Qi YANG, Beijing (CN)

(21) Appl. No.: 15/916,309

(22) Filed: Mar. 9, 2018

(30) Foreign Application Priority Data
Oct. 24, 2017 (CN) 201711000319.4

(51) Int. Cl. A61B 5/021; A61B 5/0452; A61B 5/00

(52) U.S. Cl. CPC A61B 5/02125; A61B 5/0535; A61B 5/72; A61B 5/0452

(57) ABSTRACT

A method for measuring blood pressure includes: detecting a cardiac impendence signal and a pulse signal of a person to be tested, determining a starting point of time of ejection from the cardiac impendence signal, determining a reference point of time from the pulse signal, determining a transmission time of pulse wave from the starting point of time of ejection and the reference point of time, and determining blood pressure of the person to be tested from the transmission time and a time-blood pressure formula.

### CROSS REFERENCE

The present application claims the priority of Chinese Patent Application No. 201711000319.4 titled by “Method and Device for Measuring Blood Pressure” and filed on Oct. 24, 2017, and the entire contents thereof are incorporated herein by reference as part of the present application.

### TECHNICAL FIELD

The present disclosure relates to the technical field of measuring blood pressure, in particular to a method for measuring blood pressure and a device for measuring blood pressure.

### BACKGROUND

Blood pressure, which is an important physiological index, directly reflects the health state of a human body. In order to be aware of the condition of blood pressure, a special device for measuring blood pressure is used to measure blood pressure to provide data for diagnosing illnesses such hypertension.

The conventional devices for measuring blood pressure typically comprise mercury sphygmomanometers and electronic sphygmomanometers and the like, of which the mercury manometers are based on the Korotkoff-sound method, also called as the mercury auscultation method, to measure blood pressure, i.e., to determine blood pressure by listening to the original Korotkoff-sound in conjunction with the scale corresponding to the column of mercury. The conventional electronic sphygmomanometers are mainly based on oscillometric method to measure blood pressure, i.e., to determine blood pressure by sensing the oscillating wave generated by the blood flowing through the blood vessel.

However, the conventional devices for measuring blood pressure such as mercury manometers and electronic manometers all require use of the components such as an inflatable cuff to apply a pressure at the arm of a person to be tested, and a period of time between two measurements to inflate or deflate the inflatable cuff, resulting in that the conventional devices for measuring blood pressure cannot perform continuous blood measurements, and it is difficult to achieve long-term continuous monitoring of blood pressure.

It should be noted that the information disclosed in the above background section is only to enhance understanding of the background of the disclosure, and thus may contain information which is not part of the prior art known to those skilled in the art.

### SUMMARY

The disclosure has an objective to provide a method for measuring blood pressure and a device for measure blood pressure, and in turn to solve, at least to some extent, one or more problems due to limitations and deficiencies in the prior art.

In one aspect of the disclosure, there is provided a method for measuring blood pressure comprising:

detecting a cardiac impendence signal and a pulse signal of a person to be tested;

determining a starting point of time of ejection from the cardiac impendence signal;

determining a reference point of time from the pulse signal; determining a transmission time of a pulse wave from the starting point of time of ejection and the reference point of time; and determining blood pressure of the person to be tested from the transmission time and a time-blood pressure formula.

In an exemplary embodiment of the disclosure, the time-blood pressure formula is:

$$BP = \frac{1}{γ} [ ln ( \frac{ρ {dS}^{2}}{{E}_{0} h} ) - 2 ln ( PTT ) ]$$

where, BP is the blood pressure, PTT is the transmission time, γ is a reference coefficient, ρ is viscosity of blood, d is diameter of an inner wall of the blood vessel, S is a transmission distance of the pulse wave, E₀ is elasticity modulus of the blood vessel when the pressure is 0, and h is thickness of wall of the blood vessel.

In an exemplary embodiment of the disclosure, the reference point of time and the starting point of time of ejection are in the same heartbeat period.

In an exemplary embodiment of the disclosure, the transmission time is a period of time between the reference point of time and the starting point of time of ejection.

In an exemplary embodiment of the disclosure, determining the starting point of time of ejection comprises:

applying a zero-crossing-point detection to the cardiac impendence signal to obtain a zero crossing point of cardiac impendence; and

determining the starting point of time of ejection from the zero crossing point of cardiac impendence.

In an exemplary embodiment of the disclosure, determining the reference point of time comprises:

differentiating the pulse signal to obtain a pulse differential signal;

determining the reference point of time from maximum of the pulse differential signal.

In an exemplary embodiment of the disclosure, determining the reference point of time comprises:

differentiating the pulse signal to obtain a pulse differential signal;

applying a zero-crossing-point detection to the pulse differential signal to obtain a zero crossing point of pulse differentiation; and

determining the reference point of time from the zero crossing point of pulse differentiation.

In an exemplary embodiment of the disclosure, the pulse signal is within a range of predetermined threshold.

In an exemplary embodiment of the disclosure, the method for measuring blood pressure further comprises:

sending the blood pressure to a receiving terminal which is able to store the blood pressure.

In an aspect of the disclosure, there is provided a device for measure blood pressure comprising:

a detection module for detecting a cardiac impendence signal and a pulse signal of a person to be tested;

a first process circuit for determining a starting point of time of ejection from the cardiac impendence signal;

a second process circuit for determining a reference point of time from the pulse signal;

a time determination circuit for determining a transmission time of a pulse wave from the starting point of time of ejection and the reference point of time; and

a blood pressure determination circuit for determining blood pressure of the person to be tested from the transmission time and a time-blood pressure formula.

It should be understood that the above described general description and the following detailed description are only exemplary and illustrative, and not intended to limit the disclosure.

### BRIEF DESCRIPTION OF THE DRAWINGS

The accompanying drawings which are here incorporated in and constitute part of the specification show embodiments in compliance with the disclosure, and serve to explain the principle of the disclosure in conjunction with the specification. Obviously, the accompanying drawings described below involve only some embodiments of the disclosure, based on which those skilled in the art also can obtain other drawings without creative efforts.

FIG. 1 is a flow chart according to an embodiment of a method for measuring blood pressure of the disclosure.

FIG. 2 is a flow chart for determining the starting point of time of ejection according to a method for measuring blood pressure of the disclosure.

FIG. 3 is a flow chart for a first way of determining the reference point of time according to a method for measuring blood pressure of the disclosure.

FIG. 4 is a flow chart for a second way of determining the reference point of time according to a method for measuring blood pressure of the disclosure.

FIG. 5 is a flow chart according to another embodiment of a method for measuring blood pressure of the disclosure.

FIG. 6 is a block diagram according to an embodiment of a device for measuring blood pressure of the disclosure.

FIG. 7 is a block diagram of the detection module of a device for measuring blood pressure of the disclosure.

FIG. 8 is a block diagram according to another embodiment of a device for measuring blood pressure of the disclosure.

FIG. 9 is a schematic view of the principle of determining the reference point of time according to a method for measuring blood pressure of the disclosure.

FIG. 10 is a schematic view of the principle of determining the transmission time according to a method for measuring blood pressure of the disclosure.

### DETAILED DESCRIPTION

Now more detailed description will be made to the exemplary embodiments with reference to the accompanying drawings. The exemplary embodiments, however, can be performed in various ways and should be interpreted to be limited to the illustrated examples here. To the contrary, the embodiments are provided to make the disclosure more full and complete and convey the ideas thereof as a whole to those skilled in the art. The described features, structures or characteristics may be combined in one or more embodiments in any appropriate way. In following description, many details are provided to give a full understanding of the embodiments of the disclosure. However, those skilled in the art would appreciate that the technical solution of the disclosure can be practiced, omitting one or more particular details, or can use other methods, elements, devices and steps and the like. In some cases, the well-known technical solutions are not shown or described in detail in order not to reverse the primary and the secondary and to make the aspects of the disclosure indistinct.

Moreover, the accompany drawings are only for exemplary illustration of the disclosure and not necessarily drawn to scale. Same numeral references through the drawings denote same or similar parts, and detailed description therefor will be omitted. Some block diagrams in the drawings are functional entities which do not necessarily correspond to physically or logically independent entities. Those entities may be embodied in form of software, or accomplished in one or more hardware modules or ICs, or accomplished in different networks and/or processor devices and/or micro-controller devices.

The words such as “a”, “an”, “the”, “said” and “aforesaid” are used to indicate that there is one or more elements/component parts/and the like. The words such as “comprise”, “include”, “have” and “has” are used to indicate an opening meaning of including something therein and that there may be other elements/component parts/and the like in addition to the listed elements/component parts/and the like. The words such as “first”, “second” and “third” are only used for indication and not intended to limit the number of the subject.

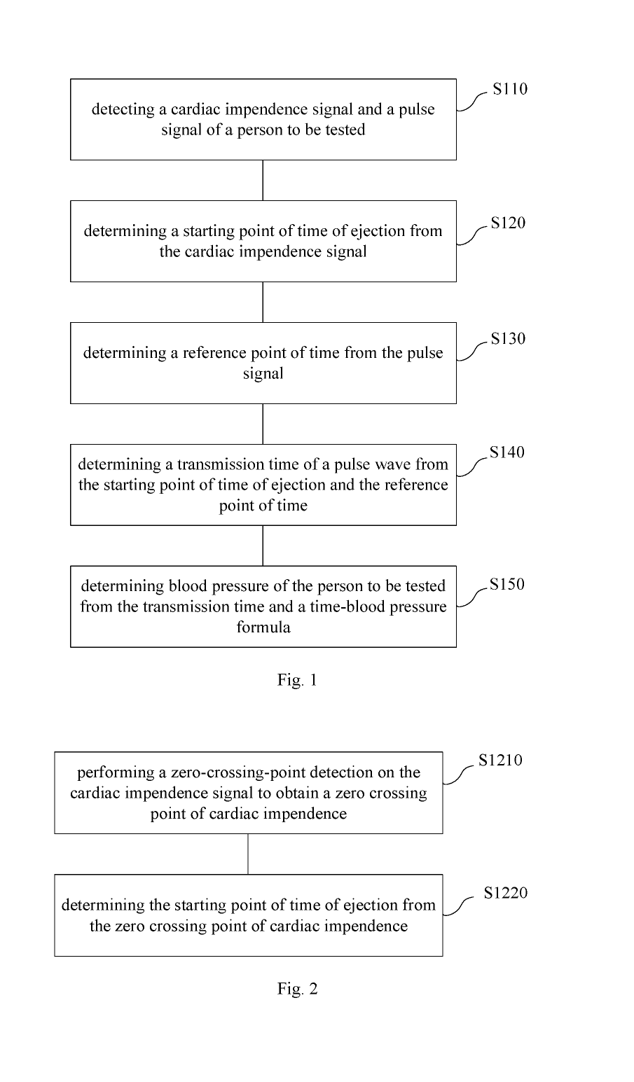

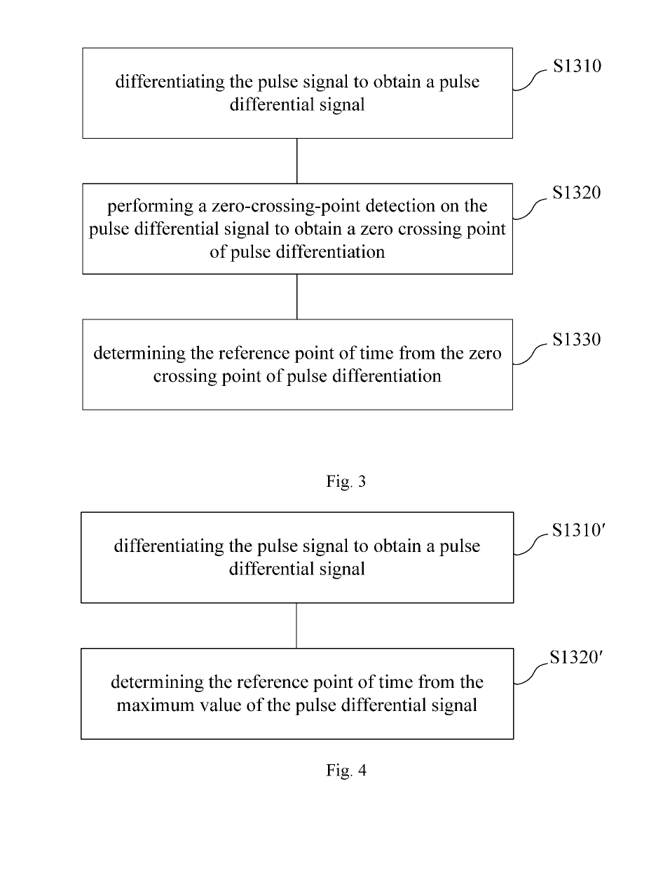

In an exemplary embodiment there is provided a method for measuring blood pressure. As shown in FIG. 1, the method for measuring blood pressure according to the exemplary embodiment may comprises:

in step S110, detecting a cardiac impendence signal and a pulse signal of a person to be tested;

in step S120, determining a starting point of time of ejection from the cardiac impendence signal;

in step S130, determining a reference point of time from the pulse signal;

in step S140, determining a transmission time of a pulse wave from the starting point of time of ejection and the reference point of time; and

in step S150, determining blood pressure of the person to be tested from the transmission time and a time-blood pressure formula.

With the method for measuring blood pressure according to the exemplary embodiment, real-timely, the cardiac impendence signal of the person to be tested can be collected and the starting point of time of ejection can be determined therefrom; at the same time, real-timely, the pulse signal of the person to be tested can be collected and the reference point of time can be determined therefrom. The transmission time of pulse wave can be determined from the starting point of time of ejection and the reference point of time. Since the transmission time of pulse wave is linearly correlated with the blood pressure, the blood pressure of the person to be tested can be determined from the transmission time and the time-blood pressure formula which reflects the relationship between the transmission time of pulse wave and the blood pressure. Accordingly, the blood pressure of the person to be tested can be measured real-timely to facilitate long-term continuous monitoring of the blood pressure of the person to be tested.

Further description will be made to the steps of the method for measuring blood pressure according to the exemplary embodiment.

In step S110, a cardiac impendence signal and a pulse signal of the person to be tested are detected.

Regarding cardiac impendence signal, a real-time detection can be made thereon by fixing an excitation electrode and a test electrode at predetermined sites of the body of the person to be tested, applying exciting signal to the person to be tested through the excitation electrode and performing a real-time detection through the test electrode. At the same time, the signal detected by the test electrode is processed by a test circuit connected with the test electrode to obtain the cardiac impendence signal. The aforesaid exciting signal may be constant current of high frequency and low amplitude, for example, a 50 KHz sinusoidal constant current of 2 mA. Both the number of the aforesaid excitement electrode and the number of the aforesaid test electrode may be one or more. The excitement electrode may be fixed at the neck of the person to be tested, and the test electrode may be fixed at the breast of the person to be tested. Of course, the fixing positions of the excitement and the test electrodes are not limited thereto, but may be at other sites which are no longer listed here.

Regarding pulse signal, the pulse of the person to be tested may be detected by an infrared pulse sensor or photoelectric pulse sensor, and subject to processes such as noise reduction and magnification, then the pulse signal can be obtained real-timely. The real-time detection of the pulse signal may also be performed by sensors such as cardiac sound pulse sensors, or other measurement devices capable of real-time detection of the pulse signal, which are not listed here.

Additionally, the pulse signal may be detected within a predetermined range of threshold such that the detected pulse signal is within the predetermined range of threshold in order to exclude oversized or undersized abnormal signal, helping improve the accuracy of detection. The predetermined range of threshold may comprise a maximum threshold and a minimum threshold, specific sizes of which are not particularly defined here. The predetermined range of threshold may be kept unchanged, or may be updated every a predetermined period of time which may be 1 second (s), 2 s and so on.

It should be noted that the ways to measure the cardiac impendence signal and the pulse signal are not limited to the above described embodiments, and other ways may be adopted, as long as the cardiac impendence signal and the pulse signal can be detected real-timely. For details, please refer to the measurements of the cardiac impendence signal and the pulse signal in the related art which are not described in detail here.

In step S120, the starting point of time of ejection is determined from the cardiac impendence signal.

The starting point of time of ejection may be a point of time corresponding to the starting point of ventricular ejection during a heartbeat period of the person to be tested. As shown in FIG. 2, exemplarily determining the starting point of time of ejection may comprise the steps S1210 and S1220, in which

in the step S1210, a zero-crossing-point detection is performed on the cardiac impendence signal to obtain the zero crossing point of cardiac impendence.

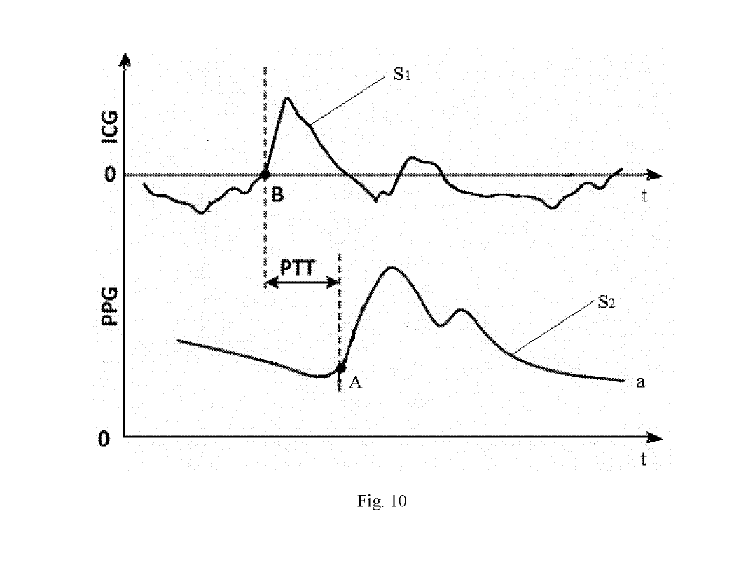

A zero-crossing-point detection circuit may be used to real-timely detect the cardiac impendence signal in order to determine the zero crossing point of cardiac impendence. As shown in FIG. 10, the zero crossing point may be the point B of the C wave in the impendence cardiogram of the cardiac impendence signal, i.e., the starting point of the ventricular ejection. For the C wave and its point B in the impendence cardiogram, please refer to the impendence cardiogram in the related art, and no details are described here. Here, no particular definition will be made for the specific configuration of the above described zero-crossing-point detection circuit, as long as the zero crossing point of cardiac impendence can be detected. For details, please refer to the existing zero-crossing-point detection circuits.

In the step S1220, the starting point of time of ejection is determined from the zero crossing point of cardiac impendence.

The point of time to which the zero crossing point of cardiac impendence corresponds may serve as the starting point of time of ejection, i.e., the starting point of time of ejection may be the point of time to which the point B of the C wave in the impendence cardiogram. Other ways may be applied to determine the starting point of time of ejection, for example, the image recognition technique may be used to recognize the starting point of time of ejection during a heartbeat period from the impendence cardiogram, which are no longer listed here.

In the step S130, the reference point of time is determined from the pulse signal.

The reference point of time may be the point of time to which a feature point in the pulse wave formed by the pulse signal corresponds. The feature point may be a peak, a valley, or a point of maximum slope of the pulse wave during a heartbeat period. Of course, it may be other points. There are a variety of ways to determine the above described reference point of time. Meanwhile, the reference point of time and the above described starting point of time of rejection may be or may not be points of time within the same heartbeat period. Now some examples will be given.

According to a first way to determine the reference point of time, the peak or valley in a heartbeat period of the pulse wave may be considered as a feature point for determining the reference point of time. In particular, determining the reference point of time comprises the steps of S1310, S1320 and S1330.

In the step S1310, the pulse signal is differentiated to obtain a pulse differential signal.

The pulse signal may be real-timely input into a differential operation circuit by which the pulse differential signal is real-timely obtained by differentiating the pulse signal. For the specific structure of the differential operation circuit, please refer to the existing differential operation circuits which are not described in detail here. The pulse differential signal corresponds to the slope of a point in the pulse wave.

In the step S1320, a zero-crossing-point detection is performed on the pulse differential signal to obtain the zero crossing point of pulse differentiation.

The zero crossing point of pulse differentiation is a pulse differentiation signal with a value of 0. With regard to a pulse wave, the zero crossing point of pulse differentiation corresponds to a peak or valley in a heartbeat period of the pulse wave which has a slope of 0.

In the step S1330, the reference point of time is determined from the zero crossing point of pulse differentiation.

Since the pulse differentiation signal is obtained by means of differential operation on the pulse wave, the above described zero crossing point of pulse differentiation may be based on to determine the corresponding pulse signal and the corresponding point of time which is the above described reference point of time, i.e., the point of time to which the peak or valley in a heartbeat period of the pulse wave corresponds.

According to a second way to determine the reference point of time, a point in a heartbeat period of the pulse wave which has the maximum slope may be considered as the feature point for determining the reference point of time. In particular, determining the reference point of time may comprise the steps of S1310′ and S1320′.

In the step S1310′, the pulse signal is differentiated to obtain the pulse differential signal.

For the method for obtaining the pulse differential signal, please refer to the above described step S1310, which is not to described in detail here.

In the step S1320′, the reference point of time is determined from the maximum value of the pulse differential signal.

A peak demodulation circuit may be used to detect the pulse differential signal in order to obtain the maximum value of the pulse differential signal which is the slope of a point in a heartbeat period of the pulse wave having a maximum slope, i.e., of the point of which the slope is in transit from being ascending to being descending. Based thereon, the pulse differential signal and its point of time to which the point corresponds may be determined, and the point of time is considered as the reference point of time. The maximum value of the pulse differential signal may be determined by other ways which are no longer listed here.

As shown in FIG. 9, S₁ is the pulse wave of the pulse signal, S₁′ is the wave form of the pulse differential signal, points a, b and c are within the same one heartbeat period, of which the point a is the valley of the pulse wave, the point b is the point of the pulse wave with the maximum slope, and the point c is the peak of the pulse wave.

In other embodiments of the disclosure, an image recognition technique may be applied to perform image recognition on the pulse wave in order to determine the point of time to which the peak, the valley or the point of maximum slope of the pulse wave corresponds, thereby obtaining the reference point of time. Of course, the reference point of time may be determined by other ways which are to be listed one by one here.

In the step S140, the transmission time of pulse wave is determined from the starting point of time of rejection and the reference point of time.

If the reference point of time and the starting point of time of rejection are within the same heartbeat period, the transmission time may be the spaced period of time between the reference point of time and the starting point of time of rejection. For example, when the reference point of time is the point of time having maximum slope in the pulse wave, computation may be made for the absolute value of the point of time having maximum slope and the starting point of time of rejection, thereby obtaining the transmission time of pulse wave. If the reference point of time and the starting point of time of rejection are within different heartbeat periods, from the spaced period of time between the reference point of time and the starting point of time of rejection, the spaced period(s) may be subtracted.

For example, as shown in FIG. 10, S₁ is the cardiac impendence wave, the point of time of the point B is the starting point of time of rejection, S₂ is the pulse wave, the point A is the reference point of time, PTT is the transmission time of pulse wave.

In the step S150, the blood pressure of the person to be tested is determined from the transmission time of pulse wave and a time-blood pressure formula.

The above described time-blood pressure may be:

$$BP = \frac{1}{γ} [ ln ( \frac{ρ {dS}^{2}}{{E}_{0} h} ) - 2 ln ( PTT ) ]$$

where, BP is the blood pressure of the person to be tested, PTT is the transmission time, γ is a reference coefficient, ρ is viscosity of blood, d is diameter of an inner wall of the blood vessel, S is a transmission distance of the pulse wave, E₀ is elasticity modulus of the blood vessel when the pressure is 0, and h is thickness of wall of the blood vessel, in which γ, ρ, d, S, E₀, and h all may be empirical data or data determined by experiments in advance, and γ may be 0.016-0.018 mmHg. Accordingly, the transmission time TP, after determined, may be taken into the above time-blood pressure formula to calculate the blood pressure BP of the person to be tested. Since the transmission time may be real-timely determined through the above steps of S110-S140, the blood pressure of the person to be tested may be real-timely determined, and the long-term continuous measurement of blood pressure may be achieved.

In other embodiments of the disclosure, the time-blood pressure formula may alternatively be:

BP=A×PTT+B

where both A and B are constant coefficients and can be determined by fitting of experimental data, for example, A=297, B=0.839, or A=283, B=0.745. Of course, the time-blood pressure formula is not limited to the forms in the above embodiments, but may be in other forms, as long as they can reflect the relationship between the transmission time and the blood pressure, which are not listed one by one here.

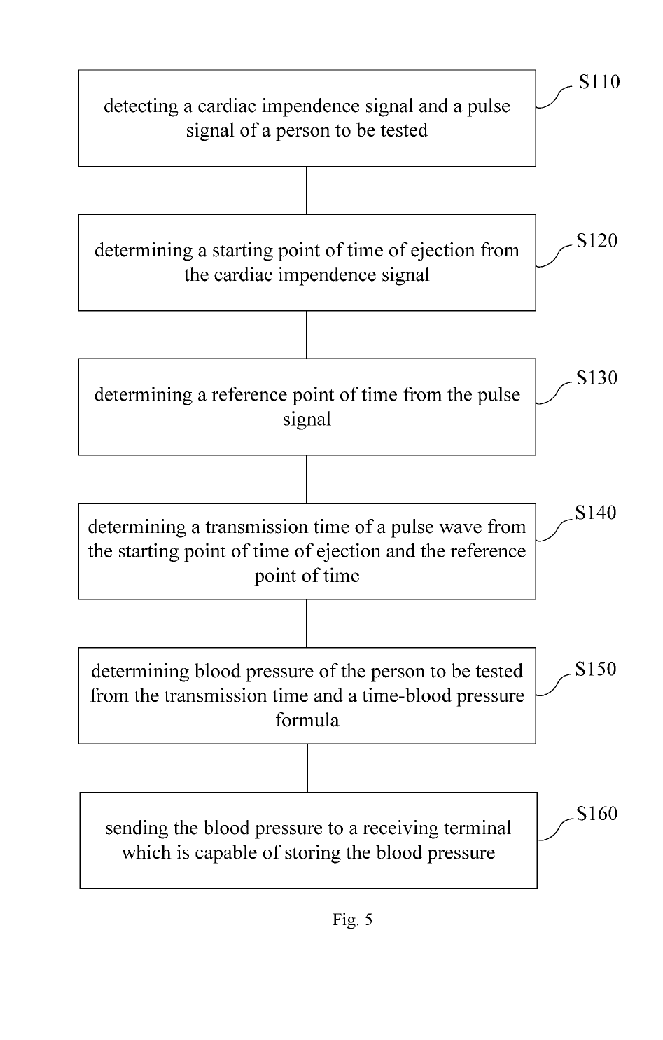

As shown in FIG. 5, in another embodiment of the disclosure, the method for measuring blood pressure, based on the above embodiments, may further comprise the step S160.

In step S160, the above described blood pressure is sent to a receiving terminal which is capable of storing the blood pressure.

In step S160, the receiving terminal may be a computer and a mobile phone and the like, and the way to send the blood pressure to the receiving terminal may be either wireless or wired way.

The following description involves device embodiments of the disclosure which may be used to perform the method embodiments of the disclosure. For details not disclosed in the device embodiments of the disclosure, please refer to the method embodiments of the disclosure.

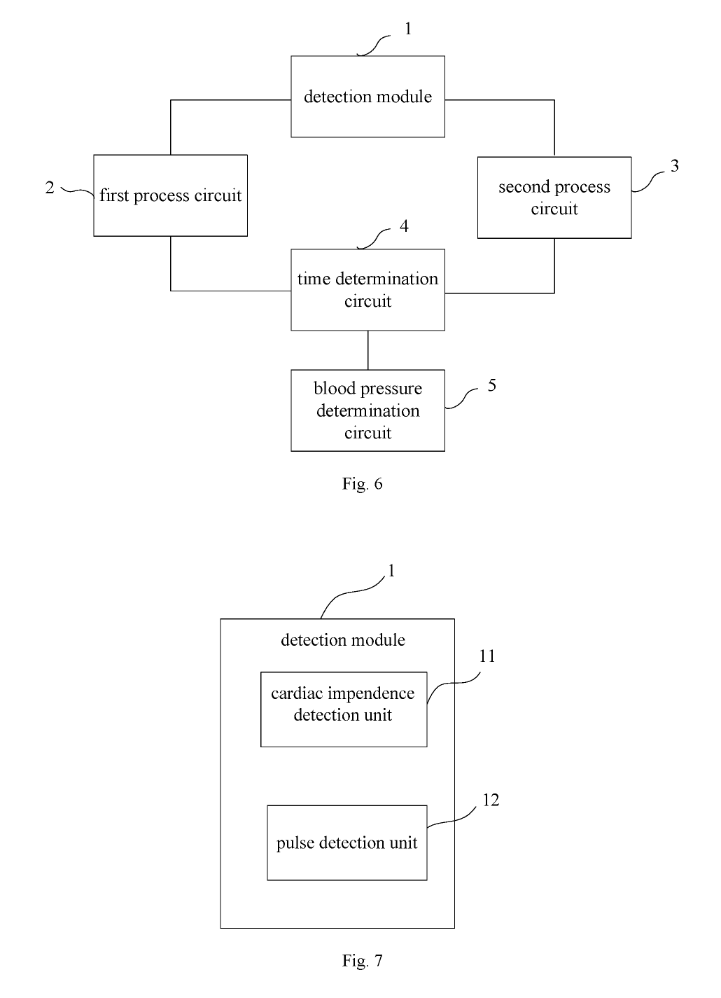

The exemplary embodiment also provides a device for measuring blood pressure which, as shown in FIG. 6, may comprise a detection modular 1, a first process circuit 2, a second process circuit 3, a time determination circuit 4, and a blood pressure determination circuit 5.

In the exemplary embodiment, the detection module 1 may be used for detecting cardiac impendence signal and pulse signal of a person to be tested. As shown in FIG. 7, the detection module 1 may comprise a cardiac impendence detection unit 11 and a pulse detection unit 12, in which:

The cardiac impendence detection unit 11 may be used to detect the cardiac impendence of the person to be tested and obtain a cardiac impendence signal. For example, the cardiac impendence detection unit 11 may comprise an excitation electrode, a test electrode for and a test circuit. An exciting signal may be applied to the person to be tested by the excitation electrode, and detected by the test electrode. Then the cardiac impendence signal may be obtained after the signal detected by the test electrode is processed by the detection circuit.

The pulse detection unit 12 may be used to detect the pulse of the person to be tested and obtain a pulse signal. For example, the pulse detection unit 12 may use a photoelectric pulse sensor or an infrared pulse sensor and the like for the detection to obtain the pulse signal.

In the exemplary embodiment, the first process circuit 2 may be used to determine the starting point of time of rejection from the cardiac impendence signal.

In the exemplary embodiment, the second process circuit 3 may be used to determine the reference point of time from the pulse signal.

In the exemplary embodiment, the time determination circuit 4 may be used to determine the transmission time of pulse wave from the starting point of time of rejection and the reference point of time.

In the exemplary embodiment, the blood pressure determination circuit 5 may be used to determine the blood pressure of the person to be tested from the transmission time and a time-blood pressure formula.

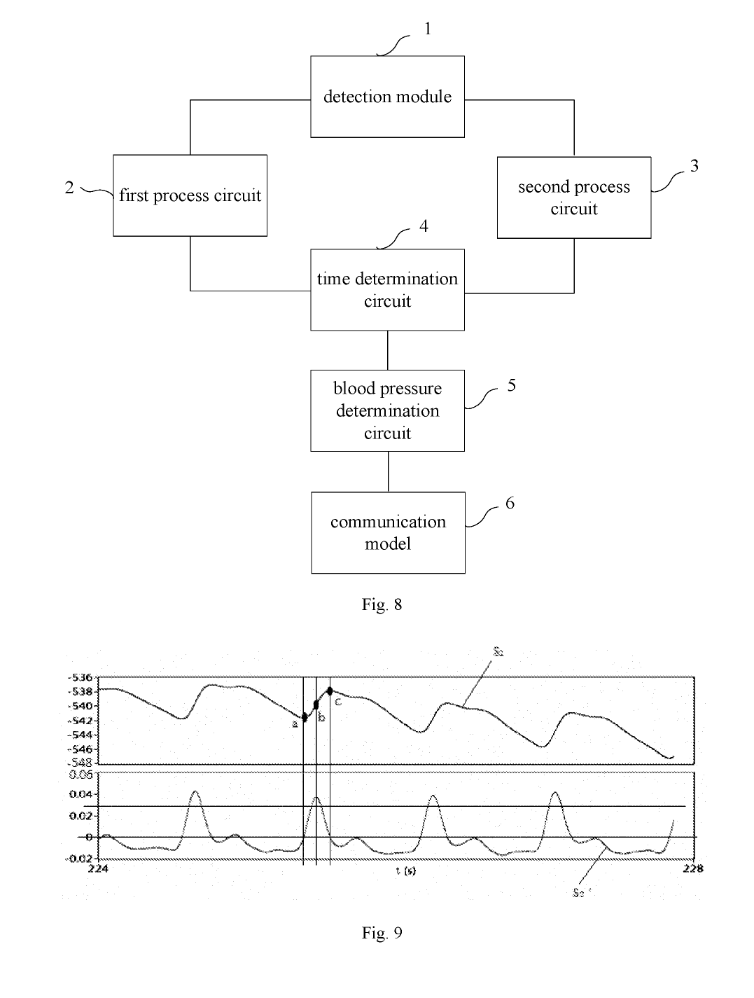

As shown in FIG. 8, in another embodiment of the disclosure, the device for measuring blood pressure, based on the above embodiment, may further comprise a communication module 6 which is a wireless or wired communication module and may comprise a transceiver component and a control component and the like. For details, please refer to the existing wireless communication modules, and no detailed description therefor are made here. The communication module 6 may communicate in a wireless communicating way with a receiving terminal such as a mobile phone and a computer, and send the measured blood pressure to the receiving terminal for storage. The communication module 6 may alternatively use a device communicating with the receiving terminal through a wired communicating way.

The details for the respective modules or units of the device for measuring blood pressure have been described in detail with reference to the corresponding method for measuring blood pressure, and are not repeated here.

It should be noted that although in the above detailed description several modules or units of the device for performing actions are mentioned, such a division is not compulsory. Instead, according to the embodiments of the disclosure, two or more of the described modules or units may have their features and functions embodied in one module or unit. Alternatively, in contrast, one of the described modules or units may have its feature and function further divided to be embodied by a plurality of modules or units.

Moreover, although the respective steps of the methods of the disclosure are described in a particular order in the figures, it is not required or implied that the steps must be performed in the particular order, or the desired result cannot be achieved until all of the shown steps have been performed. In addition or alternatively, some of the steps may be omitted, some of the steps may be combined into a single step to be performed, and/or, a single step may be split into a plurality of step to be performed, and so on.

It should be apparent to those skilled in the art from the description for the above embodiments that the described exemplary embodiments may be carried out by software or by software in combination with necessary hardware. Accordingly, the technical solutions of the disclosure may be embodied in form of software products which may be stored in nonvolatile storage media such as CD-ROM, U disks and mobile hard disks, or in network, and comprise commands to allow a computing device such as a personal computer, a sever, a mobile terminal or a network facility to carry out the methods according to the embodiments of the disclosure.

It should be apparent for those skilled in the art to envisage other embodiments of the disclosure after considering the specification and practicing the invention disclosed here. It is intended that the application encompasses any alteration, usage or adaptive change of the disclosure which follows the general principle of the disclosure and may comprise the common knowledge or customary technical means in the art which may have not been disclosed in the disclosure. The specification and the embodiments should be considered to be exemplary only. The true scope and spirit of the disclosure is indicated by the appended claims.

What is claimed is:

1. A method for measuring blood pressure comprising:
detecting a cardiac impendence signal and a pulse signal of a person to be tested;
determining a starting point of time of ejection from the cardiac impendence signal;
determining a reference point of time from the pulse signal;
determining a transmission time of pulse wave from the starting point of time of ejection and the reference point of time; and
determining blood pressure of the person to be tested from the transmission time and a time-blood pressure formula.

2. The method for measuring blood pressure according to claim 1, wherein the time-blood pressure formula is:
$$BP = \frac{1}{γ} [ ln ( \frac{ρ {dS}^{2}}{{E}_{0} h} ) - 2 ln ( PTT ) ]$$
where, BP is the blood pressure, PTT is the transmission time, γ is a reference coefficient, ρ is viscosity of blood, d is diameter of an inner wall of the blood vessel, S is a transmission distance of the pulse wave, E₀ is elasticity modulus of the blood vessel when the pressure is 0, and h is thickness of wall of the blood vessel.

3. The method for measuring blood pressure according to claim 1, wherein the reference point of time and the starting point of time of ejection are in a same heartbeat period.

4. The method for measuring blood pressure according to claim 3, wherein the transmission time is a period of time between the reference point of time and the starting point of time of ejection.

5. The method for measuring blood pressure according to claim 1, wherein the step of determining the starting point of time of ejection comprises:
applying a zero-crossing-point detection to the cardiac impendence signal to obtain a zero crossing point of cardiac impendence; and
determining the starting point of time of ejection from the zero crossing point of cardiac impendence.

6. The method for measuring blood pressure according to claim 2, wherein the step of determining the starting point of time of ejection comprises:
applying a zero-crossing-point detection to the cardiac impendence signal to obtain a zero crossing point of cardiac impendence; and
determining the starting point of time of ejection from the zero crossing point of cardiac impendence.

7. The method for measuring blood pressure according to claim 3, wherein the step of determining the starting point of time of ejection comprises:
applying a zero-crossing-point detection to the cardiac impendence signal to obtain a zero crossing point of cardiac impendence; and
determining the starting point of time of ejection from the zero crossing point of cardiac impendence.

8. The method for measuring blood pressure according to claim 4, wherein the step of determining the starting point of time of ejection comprises:
applying a zero-crossing-point detection to the cardiac impendence signal to obtain a zero crossing point of cardiac impendence; and
determining the starting point of time of ejection from the zero crossing point of cardiac impendence.

9. The method for measuring blood pressure according to claim 1, wherein the step of determining the reference point of time comprises:
differentiating the pulse signal to obtain a pulse differential signal;
determining the reference point of time from maximum of the pulse differential signal.

10. The method for measuring blood pressure according to claim 1, wherein the step of determining the reference point of time comprises:
differentiating the pulse signal to obtain a pulse differential signal;
applying a zero-crossing-point detection to the pulse differential signal to obtain a zero crossing point of pulse differentiation; and
determining the reference point of time from the zero crossing point of pulse differentiation.

11. The method for measuring blood pressure according to claim 9, wherein the pulse signal is within a range of predetermined threshold.

12. The method for measuring blood pressure according to claim 10, wherein the pulse signal is within a range of predetermined threshold.

13. The method for measuring blood pressure according to claim 1, further comprising:
sending the blood pressure to a receiving terminal which is configured to store the blood pressure.

14. A device for measure blood pressure comprising:
a detection module for detecting a cardiac impendence signal and a pulse signal of a person to be tested;
a first process circuit for determining a starting point of time of ejection from the cardiac impendence signal;
a second process circuit for determining a reference point of time from the pulse signal;
a time determination circuit for determining a transmission time of a pulse wave from the starting point of time of ejection and the reference point of time; and
a blood pressure determination circuit for determining blood pressure of the person to be tested from the transmission time and a time-blood pressure formula.
